(19) United States
(12) Patent Application Publication
(10) Pub. No.: US 2006/0025771 A1
(43) Pub. Date: Feb. 2, 2006

(54) Helical reverse angle guide and advancement structure with break-off extensions

(76) Inventor: Roger P. Jackson, Prairie Village, KS (US)

Correspondence Address: LAW OFFICE OF JOHN C. MCMAHON, P.O. BOX 30069, KANSAS CITY, MO, 64112, US

(21) Appl. No.: 11/246,320

(22) Filed: Oct. 7, 2005

Related U.S. Application Data
(63) Continuation-in-part of application No. 10/986,377, filed on Nov. 10, 2004.
(63) Continuation-in-part of application No. 09/644,777, filed on Aug. 23, 2000.
(60) Provisional application No. 60/627,000, filed on Nov. 10, 2004.

(51) Int. Cl. A61F 2/30

(52) U.S. Cl. 606/61

(57) ABSTRACT

A spinal fixation device combines an anchor member with an open receiver, such as a polyaxial bone screw or a hook, with a rotatable closure that operably clamps a spinal fixation rod to the anchor member. The anchor member has spaced apart arms forming a rod receiving channel. The arms have arm extensions or tabs connected to main portions of the arms by weakened regions to enable the extensions to be broken off or separated after the rod is clamped. The closure and inner surfaces of the arms and tabs have mating helical, anti-splay, reverse angle guide and advancement structure formed thereon that mechanically cooperate to prevent splaying of the arms and the extensions as the closure is advanced into the rod receiving channel. The increased length of the arms with the extensions enables the rod to be captured at a greater distance from the seat of the channel and allows the rod to be urged toward the seat by helical advancement of the closure into the channel, starting between the extensions. Separation of the break-off extensions results in an implant with a desirable low profile.

### CROSS-REFERENCE TO RELATED APPLICATIONS

This application claims the benefit of U.S. Provisional Application Ser. No. 60/627,000 filed Nov. 10, 2004, and is a continuation-in-part of U.S. patent application Ser. No. 10/986,377 filed Nov. 10, 2004, and also is a continuation-in-part of U.S. patent application Ser. No. 09/644,777 filed Aug. 23, 2000.

### BACKGROUND OF THE INVENTION

The present invention relates to improvements in interlocking or interconnecting helical guide and advancement structures such as reverse angle thread forms and helical flanges and, more particularly, to mating helical guide and advancement arrangements providing anti-splay interconnection when radial loading or engagement occurs. Such guide and advancement structures with anti-splay contours are particularly advantageous when used in combination with open headed bone screws formed with extended arms or tabs to facilitate the capture and reduction of spinal fixation rods, after which the arm extensions or tabs are broken off at weakened areas to form a low profile implant. In particular, in the present invention, the interlocking anti-splay components also are found on the extensions such that force can be applied to a closure and through the closure to a rod positioned between the extensions without splaying the extensions, as the closure holds them in fixed position relative to each other as the closure traverses between the extensions.

Medical implants present a number of problems to both surgeons installing implants and to engineers designing them. It is always desirable to have an implant that is strong and unlikely to fail or break during usage. Further, if one of a set of cooperating components is likely to fail during an implant procedure, it is desirable to control which particular component fails and the manner in which it fails, to avoid injury and to minimize surgery to replace or repair the failed component. It is also desirable for the implant to be as small and lightweight as possible so that it is less intrusive to the patient. These are normally conflicting goals, and often difficult to resolve.

One type of implant presents special problems. In particular, spinal anchor members such as bone screws, hooks, and the like are used in many types of back surgery for repair of problems and deformities of the spine due to injury, disease or congenital defect. For example, spinal bone screws typically have one end that threads into a vertebra and a receiver at an opposite end. The receiver is formed with an opening and a channel for receiving a rod or rod-like member that is then both captured in the channel and locked in the receiver to prevent relative movement between the various elements subsequent to installation.

A particularly useful type of receiver for such bone screws is an open receiver or head wherein an open, generally U-shaped channel is formed in the receiver, and the rod is simply laid in the open channel. The channel is then closed with some type of a closure member that engages the walls or arms forming the receiver and clamps or secures the rod in place within the channel.

While the open receiver devices are often necessary and preferred for usage, there is a significant problem associated with them. The open devices conventionally have two upstanding arms that are on opposite sides of the channel and receive the rod member. The top of the channel is closed by a closure member after the rod member is placed in the channel. Many open implants are closed by threaded plugs that screw into threads formed on internal surfaces between the arms, because such configurations have low profiles. However, such threaded plugs have encountered problems in that they produce radially outward forces that lead to splaying of the arms or at least do not prevent splaying that in turn may lead to loosening of parts and failure of the implant. In order to lock the rod member in place, a significant force must be exerted on the relatively small plug or on a set screw of some type. The forces are required to provide enough torque to insure that the rod member is clamped or locked securely in place relative to the bone screw, so that the rod does not move axially or rotationally therein. This typically requires torques on the order of 100 inch-pounds.

Because implants with open receivers such as bone screws, hooks and the like are relatively small, the arms that extend upwardly at the receiver can be spread by radially outwardly directed forces in response to the application of the substantial torquing force required to clamp the rod member. Historically, early closures were simple plugs that were threaded with V-shaped threads and were screwed into mating threads on the inside of each of the arms. Outward flexure of the arms of the receiver was caused by mutual camming action of the V-shaped threads of the plug and receiver as advancement of the plug was resisted by clamping engagement with the rod while rotational urging of the plug continued. If the arms of such a receiver are sufficiently spread, they can allow the threads to loosen or disengage and the closure to fail. To counter this, various engineering techniques have been applied to the receiver to increase resistance to the spreading force. For example, in some receivers, the arms were significantly strengthened by increasing the width of the arms by many times. This leads to a larger profile implant, which is always undesirable and may limit the working space afforded to the surgeon during implant procedures. Alternatively, external caps have been devised that engage external surfaces of the receiver. In either case, the unfortunate outcome is a substantial increase in the bulk, size and profile of the implant, especially when external nuts have been used, that take up space along the rod, so as to leave too little space for placement of all of the implants needed for a particular procedure.

The radial expansion problem of V-threads has been recognized in various other applications of threaded joints. To overcome this problem, so-called “buttress” thread forms have been developed. In a buttress thread, the trailing or thrust surface, also known as the load flank, is oriented perpendicular to the thread axis, while the leading or clearance surface, also known as the stab flank, remains angled. This results in a neutral radial reaction of a threaded receptacle to torque on the threaded member received. However, even buttress threaded closures may fail as such do not structurally resist splaying of the arms.

Another challenge of medical implant design is the placement or capture of a rod or other structural member between the arms of an open receiver. Rods implanted in spinal fixation systems are typically bent or shaped to determine the shape of the corrected curvature of the spinal column and are anchored along their length by open receiver bone screws implanted into individual vertebrae. Because of the complex curvature that must be applied to the rods, it is often difficult to capture a portion of a straight or curved rod in a bone screw receiver and to clamp the rod within the receiver arms because such receiver arms are often minimized in length to reduce the profile thereof and minimize the impact of the implanted system on the patient. So although it is desirable, on the one hand, to form the arms of an open receiver as short as possible to result in a low profile implant, it is often difficult to urge a spinal fixation rod into the U-shaped channel between the arms of such a receiver.

### SUMMARY OF THE INVENTION

The present invention solves one or more problems previously described herein by combining a reverse angle structure for guiding and advancing a closure member into a receiver with the addition of arm extensions or tabs. Such extensions are disposed adjacent to main portions of the arms and connected thereto by weakened break-off regions.

As compared to buttress and square thread forms that have a neutral radial effect on the screw receptacle, Applicant's reverse angle structure of the invention provides a thread form that positively draws threads of a receiver radially inwardly toward the thread axis when a closure member is rotated and torqued therein. In a reverse angle thread form, the trailing side of the external thread is angled toward the thread axis instead of away from the thread axis, as in conventional V-threads. The present invention utilizes such a thread form to provide an improved mating guide and advancement reverse angle structure for guiding and advancing a closure member between both the arm extensions and the receiver arms in response to relative rotation of the closure member and the receiver. The extended arms of the receiver provide ease in capturing a rod or other structural member therebetween. A closure member may then be more easily inserted and rotated to drive the rod downwardly into the receiver of the implant. Extensions according to the invention necessarily include weakened regions, providing a break-off location for removal of the extensions after the closure is fully seated in the implant, resulting in a desired low profile implant.

The reverse angle guide and advancement structure of the present invention provides a distinct advantage over the use of conventional V-shaped threads in which the potential for outward flexure and splaying of the extensions, as well as the receiver arms, would be great, and might further result in the undesirable break off of the extensions prior to the closure member being disposed in the receiver, unless some sort of cap or sleeve would be used to keep the extensions from splaying. According to the invention, inner surfaces of the extensions have a helical reverse angle guide and advancement structure formed thereon to receive a closure with a complementary reverse angle guide and advancement structure thereon for rotation into the arms of the receiver. Stated in another way, the extensions have the same anti-splay structure thereon as is found on the arms of the receiver. Furthermore, the reverse angle structure on the extensions is aligned with that on the arms so as to provide a continuous helical path for the mating structure on the closure member to follow.

The extensions or tabs enable the rod to be captured at a greater distance from the anchoring vertebra and urged toward the vertebra by advancement of the closure toward the open receiver. Just as the anti-splay guide and advancement structure on the closure member and the receiver arms cooperate to prevent splaying of the arms, the anti-splay structure on the extensions cooperates with the cooperating structure on the closure to prevent unwanted splaying of the extension and guides the closure to allow mating with the guide and advancement structure on the arms simply by rotating the closure. Thus, the guide and advancement structure on the closure does not have to be realigned with the cooperating structure on the arms. Furthermore, pressure applied to the rod while between the extensions is continued as the rod passes between the arms. The anti-splay reverse angle structure of the present invention makes the use of such extended arms or tabs possible, even when substantial force must be applied to the rod and even though the extensions include weakened regions so that when a rod has been seated in the rod receiving channel of the receiver and sufficiently clamped, the extensions or tabs can be broken off the main portions of the arms to provide the desired low profile implant. Because of the flimsy or weakened nature of such extensions, it would not even be feasible to successfully equip extensions with V-threads, not only because of the potential for outward splaying of the extensions as force is applied to the rod by the closure member, but also because of the potential that such splaying would cause premature break-off of such extensions.

### Objects and Advantages of the Invention

Objects and advantages of the present invention include: providing an improved helical guide and advancement structure for guiding and advancing an inner member into an outer member; providing a reverse angle structure wherein the outer member is subject to being splayed in reaction to advancement and torquing of the inner member within the outer member and wherein the inner member and the outer member are particularly configured to cooperate in such a manner as to radially resist such splaying while allowing rotation and axial advancement; providing such a reverse angle structure for cooperative radially overlapping surfaces between a closure and an implant with open receiver arms equipped with extensions for receiving a rod being passed between the extensions to receiver arms, the closure pressing against the rod by the rotation of the closure along the extension and the arms; providing such a reverse angle guide and advancement structure that is particularly well adapted for use in surgically implanted structure, such as spinal fixation hardware and, particularly, to receivers and cooperating closures that are used to receive and clamp spinal fixation rods; providing such a reverse angle guide and advancement structure that is particularly well adapted for use with bone screws having open receivers with extended arms for facilitating the capture and reduction of spinal fixation rods and that are afterwards separated from the screw receiver and related implants to provide low profile implants; and providing such improved reverse angle helical guide and advancement structure that is economical to manufacture, strong and effective in use, and is particularly well adapted for the intended purpose thereof.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is an exploded perspective view of a polyaxial bone screw assembly according to the present invention having a shank, a receiver with arm extensions, and a shank retaining structure and further showing a rod and closure structure.

FIG. 2 is an enlarged cross-sectional view of the shank taken along the line 2-2 of FIG. 1.

FIG. 3 is an enlarged top plan view of the shank of FIGS. 1 and 2.

FIG. 4 is an enlarged top plan view of the retaining structure of FIG. 1.

FIG. 5 is an enlarged cross-sectional view taken along the line 5-5 of FIG. 4.

FIG. 6 is a cross-sectional view of the receiver taken along the line 6-6 of FIG. 1 and showing the retaining structure seated in the receiver, also in cross-section, and illustrating the retaining structure being inserted into the receiver in dashed lines.

FIG. 7 is an enlarged and fragmentary side elevational view of the assembly of FIG. 1 with portions broken away to show the detail thereof, illustrating the retaining structure mated with the shank and the closure structure pressing upon a rod disposed between the arm extensions and reducing the rod toward the receiver by rotation of a tool engaged with the closure structure.

FIG. 8 is an enlarged and fragmentary view similar to FIG. 7 showing the rod engaged with both the shank and the closure structure and with the arm extensions removed.

FIG. 9 is a cross-sectional view taken along the line 9-9 of FIG. 8.

FIG. 10 a fragmentary and enlarged perspective view of the assembly of FIG. 1 shown completely assembled with the rod and closure structure.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Referring to the drawings in more detail, the reference numeral 1 designates a receiver according to the invention having a component of a helical guide and advancement reverse angle structure, generally 3, in combination with upwardly extending break-off tabs or extensions 5 used in conjunction with a medical implant assembly, generally 7, that embodies the present invention. It is noted that any reference to the words top, bottom, up and down, and the like, in this application refers to the alignment shown in the various drawings, as well as the normal connotations applied to such devices, and is not intended to restrict positioning of the receiver 1 and the medical implant assembly 7 in actual use.

The reverse angle guide and advancement structure 3 according to the invention includes a reverse angle thread form 10 extending helically on an inner member 16 and a complimentary reverse angle thread form 19 extending helically within an outer member 21 illustrated in the drawings as being a portion of the receiver 1. The reverse angle thread forms 10 and 19 cooperate to helically guide the inner member 16 into the outer member 21 when the inner member 16 is rotated and advanced into the outer member 21. The inner and outer thread forms 10 and 19 provide respective anti-splay surfaces 24 and 26 that cooperate to prevent splaying tendencies of the outer member 21 when the inner member 16 is strongly torqued therein.

In the illustrated embodiment the medical implant assembly 7 includes the bone screw receiver 1 embodying the outer member 21, and further includes a shank 34 having a body 36 integral with an upper portion or capture structure 38 and a retaining structure 42. The shank 34, the receiver 1 and the retaining structure 42 preferably are assembled prior to implantation of the shank body 36 into a vertebra 45.

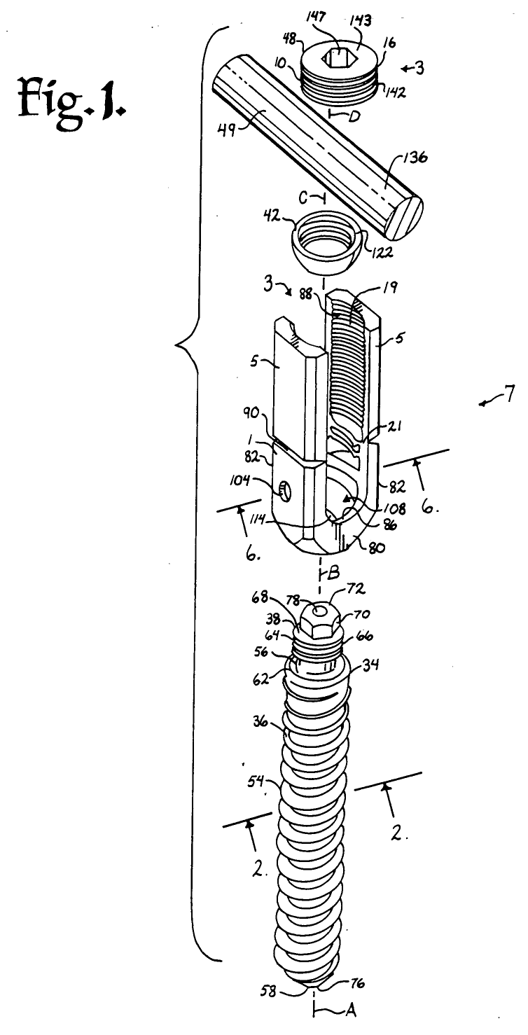

FIG. 1 further shows the illustrated inner member 16 as part of a closure structure 48 that is helically advanced by rotation thereof into the receiver 1 and torqued against a longitudinal member, such as a rod 49, to clamp the rod 49 within the receiver 1. Although embodiments of the outer member 21 and the inner member 16 are illustrated herein as the receiver 1 and the closure 48, the reverse angle structure 3 is not intended to be limited to such an application. It is especially noted that the implant assembly 7 may be a hook or other implant structure having a receiving channel for a rod or other structural member. Also, while the illustrated implant assembly 7 is shown as a polyaxial assembly, it is intended that the reverse angle structure 3 be adaptable for use with other types of polyaxial assemblies as well as mono-axial bone screws, hooks, and other types of implants.

As will be described in greater detail below, the closure structure 48 biases the rod 49 or other longitudinal member against the upper portion or capture structure 38 of the shank 34 that in turn biases the retaining structure 42 into fixed frictional contact with the receiver 1, so as to fix the rod 49 relative to the vertebra 45. The receiver 1 and the shank 34 cooperate in such a manner that the receiver 1 and the shank 34 can be secured at any of a plurality of angles, articulations or rotational alignments relative to one another and within a selected range of angles both from side to side and from front to rear, to enable flexible or articulated engagement of the receiver 1 with the shank 34 until both are locked or fixed relative to each other near the end of an implantation procedure.

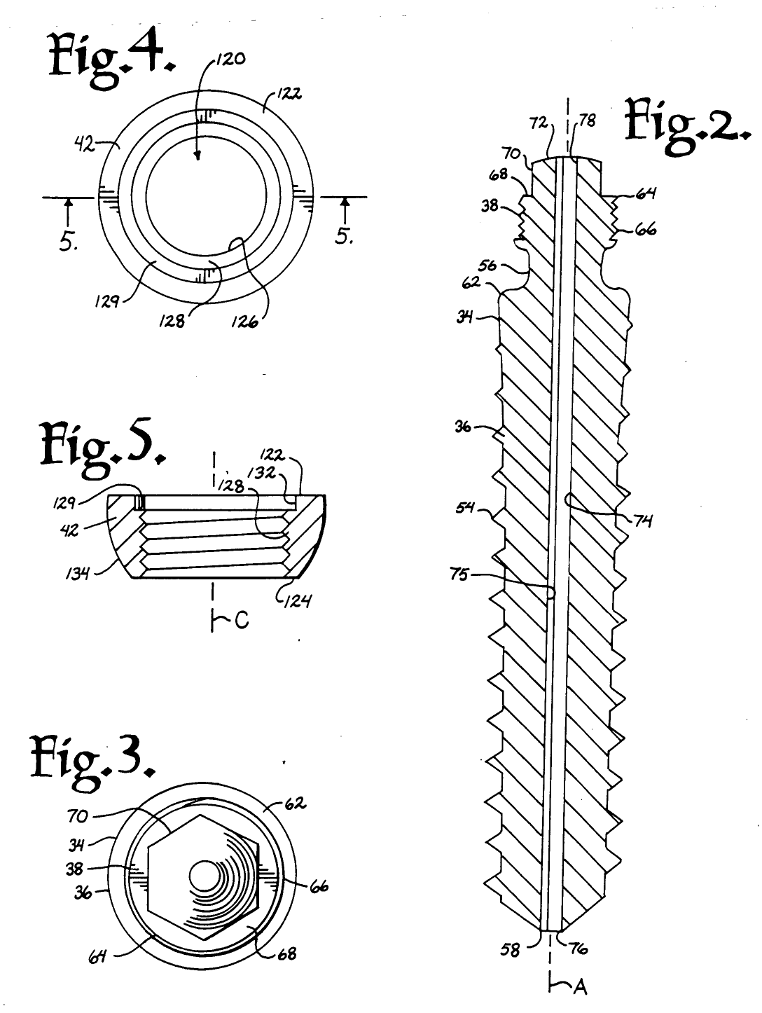

The shank 34, best illustrated in FIGS. 1-3, is elongate, with the shank body 36 having a helically wound bone implantable thread 54 extending from near a neck 56 located adjacent to the capture structure 38 to a tip 58 of the body 36 and extending radially outwardly therefrom. During use, the body 36 utilizing the thread 54 for gripping and advancement is implanted into the vertebra 45 leading with the tip 58 and driven down into the vertebra 45 with an installation or driving tool (not shown), so as to be implanted in the vertebra 45 to near the neck 56, as shown in FIGS. 8-10, and as is described more fully in the paragraphs below. The shank 34 has an elongate axis of rotation generally identified by the reference letter A.

The neck 56 extends axially outward and upward from the shank body 36. The neck 56 is of reduced radius as compared to an adjacent top 62 of the body 36. Further extending axially and outwardly from the neck 56 is the capture structure 38 that provides a connective or capture apparatus disposed at a distance from the body top 62 and thus at a distance from the vertebra 45 when the body 36 is implanted in the vertebra 45.

The capture structure 38 is configured for connecting the shank 34 to the receiver 1 and capturing the shank 34 in the receiver 1. The capture structure 38 has an outer substantially cylindrical surface 64 having a helically wound advancement structure thereon which in the illustrated embodiment is a V-shaped thread 66 extending from near the neck 56 to adjacent to a seating surface 68. Although a simple thread 66 is shown in the drawings, it is foreseen that other structures including other types of threads, such as buttress and reverse angle threads, and non threads, such as helically wound flanges with interlocking surfaces, may be alternatively used in alternative embodiments of the present invention.

The shank 34 further includes a tool engagement structure 70 disposed near a top end surface or dome 72 thereof for engagement of a driving tool (not shown) that includes a driving structure in the form of a socket. The driving tool is configured to fit about the tool engagement structure 70 so as to form a socket and mating projection for both driving and rotating the shank body 36 into the vertebra 45. Specifically in the embodiment shown in FIGS. 1-10, the tool engagement structure 70 is in the shape of a hexagonally shaped extension head coaxial with both the threaded shank body 36 and the threaded capture structure 38.

The top surface 72 of the shank 34 is preferably curved or dome-shaped as shown in the drawings, for positive engagement with the rod 49, when the bone screw assembly 7 is assembled, as shown in FIGS. 8-10 and in any alignment of the shank 34 relative to the receiver 1. In certain embodiments, the surface 72 is smooth. While not required in accordance with practice of the invention, the surface 72 may be scored or knurled to further increase frictional engagement between the surface 72 and the rod 49.

The shank 34 shown in the drawings is cannulated, having a small central bore 74 extending an entire length of the shank 34 along the axis A. The bore 74 is defined by an inner cylindrical wall 75 of the shank 4 and has a first circular opening 76 at the shank tip 58 and a second circular opening 78 at the top surface 72. The bore 74 is coaxial with the threaded body 36 and the capture structure outer surface 64. The bore 74 provides a passage through the shank 34 interior for a length of wire (not shown) inserted into the vertebra 45 prior to the insertion of the shank body 36, the wire providing a guide for insertion of the shank body 36 into the vertebra 45.

Referring to FIGS. 1 and 6 through 10, the receiver 1 has a generally U-shaped appearance with a partially cylindrical inner profile and a faceted outer profile. The receiver 1 includes a somewhat spherical base 80 integral with a pair of upstanding arms 82 forming a U-shaped cradle and defining a U-shaped channel 84 between the arms 82 with a lower seat 86 having substantially the same radius as the rod 49 for operably snugly receiving the rod 49.

Referring particularly to FIGS. 1, 6 and 7, the receiver 1 is provided with the break-off extensions or arm tabs 5 to increase the initial length of the arms 82 and, thus, forming a rod receiving passageway between the extensions 5 and thereby increasing the length of the rod receiving channel 84 by the length of the passageway. The purpose for the lengthened channel 84 is to enable capture of the rod 49 within the channel 84 at a greater distance from the vertebra 45, whereby the rod 49 can be captured by the closure structure 48 and be “reduced” or urged toward a seated position within the channel 84 by advancement of the closure 48. This provides effective leverage in reducing the position of the rod 49 or the vertebra itself. For this purpose, inner surfaces 88 of the extensions or tabs 5 are provided with the reverse angle thread 19 that extends continuously from main portions of the arms 82 and along the extensions 5 to form a continuous and uniform helical pathway therebetween.

A pair of weakened regions 90 is disposed between the arm main portions 82 and the break-off extensions 5. The weakened regions 90 may be regions adjacent v-shaped indentations or notches extending generally perpendicular to the axis A as illustrated in FIGS. 1, 6 and 7, or any other type of diminishing or lessening in the arm thickness to provide for ready separation of the extension 5 from the arms 82 by breaking the extensions 5 off of the arms 82 at the weakened regions 90. The weakened regions 90 are strong enough to enable the rod 49 to be urged toward its seated position (FIGS. 8 and 9). However, the extensions 5 can be broken off or separated from the main portions of the arms 82 by pivoting or bending the extensions 5 back and forth about the regions 90 while the main portions of the arms are held in place, after the closure structure 48 has passed between the extensions 5. The resulting low-profile implanted structure is shown in FIGS. 8-10.

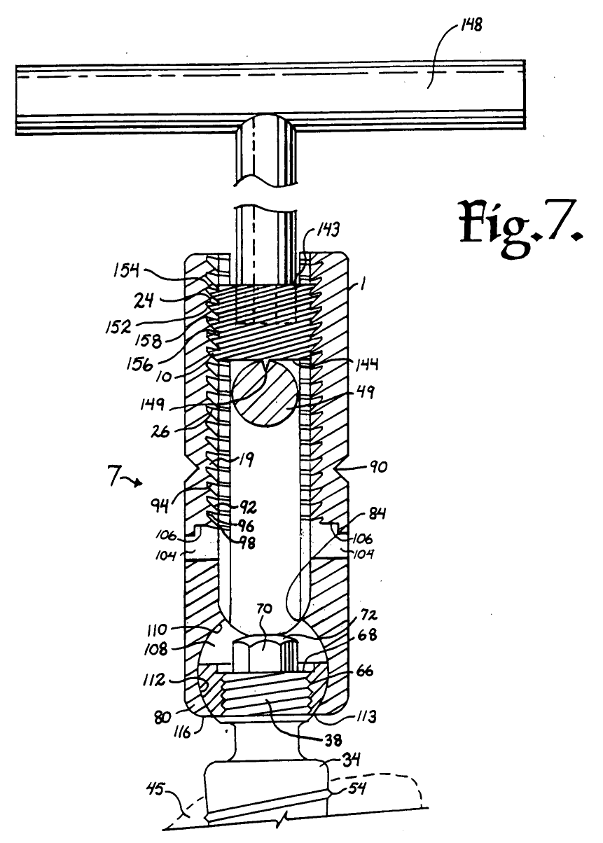

The reverse angle thread form 19 is disposed about the inner surface 88 of the extensions 5 and the arms 82 in a discontinuous generally helical pattern or configuration, which is typical of threads and can have various pitches, be counterclockwise advanced or vary in most of the ways that conventional threads vary. The thread form 19 has a leading surface 92 and a trailing surface 94 that has also been identified previously herein as the anti-splay surface 26. As used herein the terms leading and trailing refer to the direction of advancement with respect to mating engagement with the closure structure 48 when used to close the receiver 1 by moving the closure structure in a direction along a central axis of rotation B of the receiver 1 toward the base 80 of the receiver 1. In the illustrated embodiment, advancement is produced by clockwise rotation of the closure structure 48. As can be seen in FIGS. 6 and 7, the general shape of the cross section of the thread 19 is that of an obtuse triangle. It can also be seen that the intersection of the leading surface 92 and the trailing surface 94 with a plane passing through the axis of rotation B, shows that both surfaces 92 and 94 slope downwardly in a direction toward the base 80 of the receiver 1 from a root 96 to a crest 98 of the thread form 19. As compared to a buttress thread wherein the trailing surface is disposed perpendicular to the axis of rotation, in a reverse angle thread form of the invention, the trailing surface is disposed at an angle with respect to the axis of rotation, the surface sloping in generally the same direction as the leading surface. This also contrasts with convention V-threads wherein the leading and trailing surfaces slope in opposed directions. The intersection of the trailing surface 94 with a plane passing through the axis of rotation B is typically at an angle of from about 1° to about 45° relative to a line perpendicular to the axis of rotation B. Further details regarding reverse angle threads of the invention are described in U.S. patent application Ser. No. 09/644,777, filed Aug. 23, 2000, incorporated by reference herein.

Tool engaging apertures 104 are formed on outer surfaces or facets of the arms 82. The apertures 104 may be used for holding the receiver 1 during assembly with the shank 34 and the retaining structure 42 and also during the implantation of the shank body 36 into the vertebra 45. Communicating with the apertures 104 are upwardly projecting, hidden inner recesses 106. A holding tool (not shown) is sized and shaped to have structure to mate with and to be received in the aperture 104 and locked into place by pulling the holding tool slightly axially upward relative to the base 80 and toward the arm extensions 5. The holding tool and respective apertures 104 may be configured for a variety of engagement orientations, including, but not limited to, a twist on/twist off or a snap on/snap off engagement wherein the holding tool has legs that splay outwardly to position the tool for engagement in the apertures 104 and recesses 106. It is noted that the apertures 104 and the cooperating holding tool may be configured to be of a variety of sizes and locations along any of the surfaces of the arms 82.

Communicating with and located beneath the U-shaped channel 84 of the receiver 1 is a chamber or cavity 108 substantially defined by an inner surface 110 of the base 80, the cavity 108 opens upwardly into the U-shaped channel 84. The inner surface 110 is substantially spherical, with at least a portion thereof forming a partial internal spherical seating surface 112. The surface 112 is sized and shaped for mating with the retaining structure 42, as described more fully below.

The base 80 further includes a restrictive neck 113, having a radius smaller than a radius of the spherical surface 110. The neck 113 defines a bore 114 communicating with the cavity 108 and a lower exterior 116 of the base 80. The bore 114 is coaxially aligned with respect to the rotational axis B of the receiver 1. The neck 113 and associated bore 114 are sized and shaped to be smaller than a radial dimension of the retaining structure 42, as will be discussed further below, so as to form a restriction at the location of the neck 113 relative to the retaining structure 42, to prevent the retaining structure 42 from passing from the cavity 108 and out into the lower exterior 116 of the receiver 1 when the retaining structure 42 is seated within the receiver 1. However, it is foreseen that the retaining structure could be compressible (such as where such structure has a missing section) and that the retaining structure could be loaded through the neck 113 and then allowed to expand and fully seat in the spherical seating surface of the receiver 1.

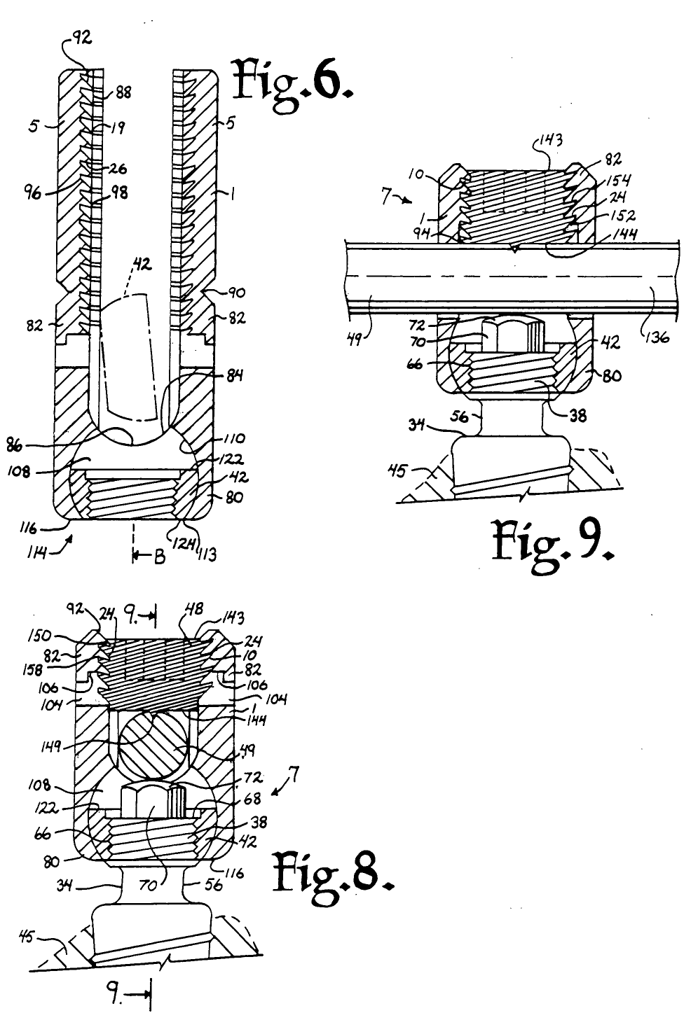

The retaining structure or ring 42 is used to retain the upper portion or capture structure 38 of the shank 34 within the receiver 1. The retaining structure 42, best illustrated by FIGS. 4-5, has an operational central axis that is the same as the elongate axis A associated with the shank 34, but when the retaining structure 42 is separated from the shank 34, the axis of rotation is identified as axis C, as shown in FIG. 5. The retaining structure 42 has a central bore 120 that passes entirely through the retaining structure 42 from a top surface 122 to a bottom surface 124 thereof. A first inner cylindrical surface 126 defines a substantial portion of the bore 120, the surface 126 having a helically wound guide and advancement structure thereon as shown by a helical rib or thread 128 extending from adjacent the bottom surface 124 to adjacent a flat, seating surface 129 disposed perpendicular to the inner surface 126.

Although a simple helical rib 128 is shown in the drawings, it is foreseen that other helical structures including other types of threads, such as buttress and reverse angle threads, and non threads, such as helically wound flanges with interlocking surfaces, may be alternatively used in an alternative embodiment of the present invention. The inner cylindrical surface 126 with helical rib 128 are configured to mate under rotation with the capture structure outer surface 64 and helical guide and advancement structure or thread 66, as described more fully below.

The retaining structure 42 further includes a second inner wall or cylindrical surface 132, coaxial with the first inner cylindrical surface 126. The surface 132 is disposed between the seating surface 129 and the top surface 122 of the retaining structure 42 and has a diameter greater than that of the cylindrical surface 126. As will be described more fully below, the cylindrical surface 132 in cooperation with the seating surface 129 and the surface 68 of the capture structure 38, provide a recess about the base of the tool engagement structure 70 and a stable seating surface for the tool (not shown) used to drive the shank body 36 into bone. The surface or wall 132 which is the outer wall of the recess may be shaped to fit an outer surface of such a driving tool and may be faceted, for example, hexagonal in shape, to better grip the driving tool.

The retaining structure or ring 12 has a radially outer partially spherically shaped surface 134 sized and shaped to mate with the partial spherically shaped seating surface 112 of the receiver 1 and having a radius approximately equal to the radius associated with the surface 112. The retaining structure radius is larger than the radius of the neck 113 of the receiver 1. Although not required, it is foreseen that the outer partially spherically shaped surface 134 may be a high friction surface such as a knurled surface or the like.

The elongate rod or longitudinal member 49 that is utilized with the assembly 7 can be any of a variety of implants utilized in reconstructive spinal surgery, but is normally a cylindrical elongate structure having a cylindrical surface 136 of uniform diameter and having a generally smooth surface. The rod 49 is preferably sized and shaped to snugly seat near the bottom of the U-shaped channel 84 of the receiver 1 and, during normal operation, is positioned slightly above the bottom of the channel 84 at the lower seat 86. In particular, the rod 49 normally directly or abutingly engages the shank top surface 72, as shown in FIGS. 8 and 9 and is biased against the dome shank top surface 72, consequently biasing the shank 34 downwardly in a direction toward the base 80 of the receiver 1 when the assembly 7 is fully assembled. For this to occur, the shank top surface 72 must extend at least slightly into the space of the channel 84 when the retaining structure 42 is snugly seated in the lower part of the receiver cavity 108. The shank 34 and the retaining structure 42 are thereby locked or held in position relative to the receiver 1 by the rod 49 firmly pushing downward on the shank top surface 72.

With reference to FIGS. 1 and 6-10, the closure structure or closure top 48 can be any of a variety of different types of closure structures for use in conjunction with the mating structure on the main portions of the upstanding arms 82 and the arm break-off extensions 5. The illustrated closure top 48 is a cylindrically shaped plug having a generally cylindrical shaped radially outer surface 142, a flat top 143 and a substantially flat bottom 144. The closure structure 48 has an axis of rotation, generally indicated by the reference numeral D. The axis of rotation D is at the radial center of the closure structure 48. An internal tool engagement structure in the form of an aperture or bore 147 that is co-axial with the axis of rotation D extends through the top 143 and partially through the closure structure 48. The aperture 147 is poly-faceted so as to have a hexagonal cross section such that the closure structure 48 can be installed or removed with an allen type tool 148 that is engageable with the structure 48 at the aperture 147. Although a hex-shaped aperture 147 is shown in the drawings, the tool engagement structure may take a variety of tool-engaging forms, such as multi-lobular drives sold under the trademark TORX, or may include more than one aperture of various shapes, such as a pair of spaced apertures, or the like. Although a particular closure structure 48 has been illustrated herein, it is foreseen that the invention can be used in conjunction with plugs and set screws of various types and configurations. For example, the closure structure may include a break off head for insertion.

The closure structure 48 also includes structure to assist in engaging and securing the rod 49, shown as a point 149 for penetrating the rod surface 136. Although not shown, such a closure structure may further include a cutting rim and/or a roughened under surface.

The closure structure 48 outer substantially cylindrical surface 142 embodies the inner member 16 having the reverse angle thread form 10. The thread form 10 includes a leading surface 152 and a trailing surface 154 that has also been identified herein as the anti-splay surface 24. As with the description herein with respect to the receiver 1, the terms leading and trailing refer to the direction of advancement of the closure structure 48 into the receiver 1 by moving the closure structure 48 in a direction along the central axis of rotation B of the receiver 1 (also about the axis D of the structure 48) and toward the base 80 of the receiver 1. The general shape of the cross section of the thread 10 is that of an obtuse triangle. It can be seen that at the intersection of the leading surface 152 and the trailing surface 154 with a plane passing through the axis of rotation D, both surfaces 152 and 154 slope upwardly or rearwardly in a direction away from the bottom surface 144 of the closure 48 from a root 156 to a crest 158 of the thread form 10. Both surfaces 152 and 154 also slope upwardly or rearwardly in a direction away from the base 80 of the receiver 1 when the closure 48 is engaged with the receiver 1.

The reverse angle thread form is shaped and positioned so as to engage the discontinuous reverse angle thread form 19 that winds on the extensions 5 and the arms 82 to provide for rotating advancement of the closure structure 48 into the receiver 1 when rotated clockwise and, in particular, to cover the top or upwardly open portion of the U-shaped channel 84 to capture the rod 49, without splaying of the extensions 5 or the arms 82. The closure structure 48 also operably biases against the rod 49 by advancement and applies pressure to the rod 49 under torquing, so that the rod 49 is urged downwardly against the shank top end surface 72 that extends into the channel 84. Downward biasing of the shank top surface 72 operably produces a frictional engagement between the rod 49 and the surface 72 and also urges the retaining structure 42 toward the base 80 of the receiver 1, so as to frictionally seat the retaining structure external spherical surface 134 fixedly against the partial internal spherical seating surface 112 of the receiver 1, also fixing the shank 34 and retaining structure 42 in a selected, rigid position relative to the receiver 1.

It is noted that as torque is applied to the closure 48 in a clockwise manner so as to advance the closure 48 in the receiver 1 the trailing surface 154 engages and pushes against the trailing surface 94 of the thread 19 of the receiver 1. The force exerted on the closure 48 by this process is countered by a reactive force acting on the receiver 1 that has a first component that is axial, that is parallel to the axis of rotation D of the closure structure 48, and a second component that has a radial inward vector, that is toward the axis of rotation D of the closure structure 48.

Prior to the polyaxial bone screw assembly 7 being placed in use according to the invention, the retaining structure 42 is typically first inserted or top-loaded, into the receiver U-shaped channel 84, as is shown in dotted lines in FIG. 6, and then into the cavity 108 to dispose the structure 42 within the inner surface 110 of the receiver 1. Then, the retaining structure 42 is rotated approximately 90 degrees so as to be coaxial with the receiver 1 and then seated in sliding engagement with the seating surface 112 of the receiver 1, also shown in FIG. 6.

With reference to FIG. 7, the shank capture structure 38 is pre-loaded, inserted or bottom-loaded into the receiver 1 through the bore 114 defined by the neck 113. The retaining structure 42, now disposed in the receiver 1 is coaxially aligned with the shank capture structure 38 so that the helical guide and advancement structure 66 rotatingly mates with the helical guide and advancement structure 128 of the retaining structure 42.

The shank 34 and or the retaining structure 42 are rotated to fully mate the structures 66 and 128 along the respective cylindrical surfaces 64 and 126, fixing the capture structure 38 to the retaining structure 42, until the seating surface 68 and the seating surface 129 are contiguous and disposed in the same plane as shown in FIGS. 7-9. Permanent, rigid engagement of the capture structure 38 to the retaining structure 42 may be further ensured and supported by the use of adhesive, a spot weld, deforming one or both threads with a punch or the like. At this time the shank 34 is in slidable and rotatable engagement with the receiver 1, while the capture structure 38 and the lower aperture or neck 113 of the receiver 1 cooperate to maintain the shank body 36 in rotational relation with the receiver 1. Only the retaining structure 42 is in slidable engagement with the head spherical seating surface 112. Both the capture structure 38 and threaded portion of the shank body 36 are in spaced relation with the receiver 1.

The assembly 7 is then typically screwed into a bone, such as the vertebra 45, by rotation of the shank 34 using a driving tool (not shown) that operably drives and rotates the shank 34 by engagement thereof with the hexagonally shaped extension head 70 of the shank 34. Preferably, when the driving tool engages the tool engagement structure or head 70, an end portion thereof is disposed in a recess defined by the structure 70, the seating surface 68, the contiguous seating surface 129 and the inner cylindrical surface 132, with a bottom surface of the driving tool contacting and frictionally engaging both the seating surface 68 and the seating surface 129. Some frictional engagement between an outer surface of the driving tool with the cylindrical surface 132 may also be achievable during rotation of the driving tool.

Typically, the receiver 1 and the retaining structure 42 are assembled on the shank 34 before inserting the shank body 36 into the vertebra 45, but in certain circumstances, the shank body 36 can be first partially implanted with the capture structure 38 extending proud to allow assembly with the receiver 1 utilizing the retaining structure 42. Then the shank body 36 can be further driven into the vertebra 45.

The vertebra 45 may be pre-drilled to minimize stressing the bone and have a guide wire (not shown) that is shaped for the cannula 74 inserted to provide a guide for the placement and angle of the shank 34 with respect to the vertebra 45. A further tap hole may be made using a tap with the guide wire as a guide. Then, the assembly 7 or the solitary shank 34, is threaded onto the guide wire utilizing the cannulation bore 74 by first threading the wire into the bottom opening 76 and then out of the top opening 78. The shank 34 is then driven into the vertebra 45, using the wire as a placement guide.

With reference to FIGS. 7-10, the rod 49 is eventually positioned between the break-off extensions 5 and the closure structure 48 is then inserted into and advanced between the extensions 5 by mating the thread 10 with the thread 19, and rotating the structure 48 downwardly toward the base 80 so as to bias or push against the rod 49. Pressure applied to the rod 49 by the structure 48 is continued as the rod 40 passes from the extensions 5 to a position disposed between the receiver arms 82 and near the seat 86. The anti-splay reverse angle structure 3 of the cooperating closure structure 48, the break-off extensions 5 and the arms 82, bias the extensions 5 and arms 82 toward one another as the closure structure 48 travels downwardly toward the base 80 of the receiver 1. Once both the rod and the closure structure 48 are disposed in the receiver 1 between the arms 82, the break-off extensions 5 may be removed by bending the extensions 5, causing the extensions 5 to break away from the arms 82 at the weakened regions 90. The closure structure 48 may then be further tightened against the rod 49 as desired.

The shank top end surface 72, because it is rounded to approximately equally extend upward into the channel 84 approximately the same amount no matter what degree of rotation exists between the shank 34 and the receiver 1 and because the domed surface 72 is sized and shaped to extend upwardly into the U-shaped channel 84, the surface 72 is engaged by the rod 49 and pushed downwardly toward the base 80 of the receiver 1 when the closure structure 48 biases downwardly toward and onto the rod 49. The downward pressure on the shank 34 in turn urges the retaining structure 42 downward toward the receiver seating surface 112, with the retaining structure seating surface 129 in frictional engagement with the receiver seating surface 112. As the closure structure 48 presses against the rod 49, the rod 49 presses against the shank and the retaining structure 42 that is now rigidly attached to the shank 34 which in turn becomes frictionally and rigidly attached to the receiver 1, fixing the shank body 36 in a desired angular configuration with respect to the receiver 1 and the rod 49.

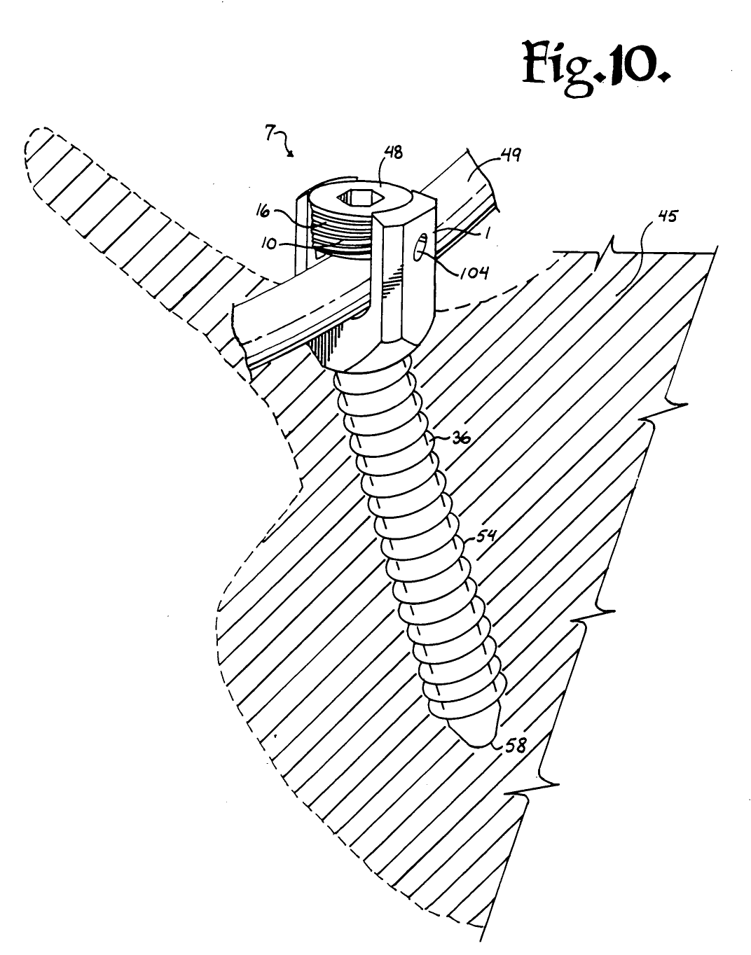

FIG. 10 illustrates the polyaxial bone screw assembly 7 and including the rod 49 and the closure structure 48 positioned in a vertebra 45. The axis A of the bone shank 34 is illustrated as not being coaxial with the axis B of the receiver 1 and the shank 34 is fixed in this angular locked configuration. Other angular configurations can be achieved, as required during installation surgery due to positioning of the rod 49 or the like.

If removal of the assembly 7 and associated rod 49 and closure structure 48 is necessary, disassembly is accomplished by using the driving tool 148 or other similarly sized tool of an Allen wrench type (not shown) mating with the aperture 147 and turned counterclockwise to rotate the closure structure 48 and reverse the advancement thereof in the receiver 1. Then, disassembly of the assembly 7 is accomplished in reverse order to the procedure described previously herein for assembly.

It is to be understood that while certain forms of the present invention have been illustrated and described herein, it is not to be limited to the specific forms or arrangement of parts described and shown.

What is claimed and desired to be secured by Letters Patent is as follows:

1. Apparatus for securing an elongate member and comprising:
(a) a receiver having spaced apart arms defining a member receiving channel therebetween, the arms having main portions and extended portions connected to the main portions by weakened regions, the main portions and the extended portions of the arms having inner surfaces;
(b) a closure sized to be received within the channel to clamp the elongate member therein;
(c) a closure guide and advancement structure extending helically about and along the closure, the guide and advancement structure having a first reverse angle thread;
(d) a discontinuous receiver guide and advancement structure extending helically about and along the inner surfaces of the main portions and the extended portions of the arms, the receiver guide and advancement structure having a second reverse angle thread being complementary and cooperatively mateable to the first reverse angle thread to prevent splaying of the arms when the closure is advanced into the receiver, the closure being advanceable against the elongate member to capture and clamp the member relative to the receiver; and
(e) the extended portions of the arms being separable from the main portions after the closure captures the elongate member within a portion of the channel defined by the main portions of the arms.

2. The apparatus of claim 1 wherein the receiver is a spinal fixation anchor and the elongate member is a spinal fixation rod.

3. The apparatus of claim 1 wherein the receiver is an open-receiver bone screw adapted for implanting in a vertebra and the elongate member is a spinal fixation rod.

4. The apparatus of claim 3 wherein the open-receiver bone screw is a polyaxial bone screw assembly comprising:
(a) a shank having a body for fixation to a bone and a capture structure extending from the body, the capture structure having an outer surface with a first helically wound thread;
(b) a receiver base integral with the receiver, the base having a seating surface partially defining a cavity, the member receiving channel communicating with the cavity, the cavity communicating with an exterior of the base through an opening sized and shaped to receive the capture structure there through; and
(c) a retaining structure having an external surface and a central bore with an internal surface having a second helically wound thread thereon, the first helically wound thread configured to rotatably mate with the second helically wound thread to secure the retaining structure to the capture structure within the receiver base cavity, the external surface configured to be in slidable mating engagement with the seating surface of the receiver base so as to enable selective angular positioning of the shank body with respect to the receiver.

5. The apparatus of claim 4 wherein:
(a) the receiver base seating surface is substantially spherical; and
(b) the retaining structure external surface is substantially spherical.

6. The apparatus of claim 4 wherein the shank is cannulated.

7. A spinal fixation structure for clamping and anchoring a spinal fixation rod and comprising:
(a) an open spinal fixation anchor including a pair of spaced apart arms defining a rod receiving channel therebetween, the arms having main portions and extended portions connected to the main portions by weakened regions, the main portions and the extended portions of the arms having inner surfaces;
(b) a closure sized to be received within the channel and adapted to be rotated and advanced to clamp a spinal fixation rod therein;
(c) a closure guide and advancement thread form extending helically about and along the closure, the thread form having a first anti-splay surface;
(d) an anchor guide and advancement thread form extending helically about and along the inner surfaces of the main portions and the extended portions of the arms, the anchor thread form having a second anti-splay surface;
(e) the first and second anti-splay surfaces being complementary and cooperating to prevent splaying of the arms when the closure is advanced into the anchor, each of the thread forms further comprising:
i) a leading surface;
ii) a trailing surface; and wherein
iii) when viewing a plane intersecting an axis of rotation of the closure with respect to the anchor, both the leading surfaces and the trailing surfaces slope in substantially the same direction with respect to a direction of advancement of the closure into the anchor; and
(f) the extended portions of the arms being separated from the main portions when the closure clamps the rod within a portion of the channel located between the main portions of the arms.

8. The structure of claim 7 wherein the anchor is a bone screw adapted for helical implanting in a vertebra, the bone screw having an open receiver.

9. The structure of claim 8 wherein the bone screw is a polyaxial bone screw.

10. In a spinal implant having a receiver with a pair of upwardly extending and spaced arms forming a rod receiving channel therebetween; the improvement comprising:
(a) upwardly extending extensions disposed adjacent to the arms, the extensions having weakened regions, the extensions bendable and removable from the arms at the weakened regions; and
(b) the arms and the extensions each having inwardly facing surfaces with discontinuous helically wound reverse angle structure thereon adapted to interlock radially with a mating reverse angle structure on a closure and to allow advancement and transfer of the closure between the extensions and the arms by rotation of the closure.
